(19) United States
(12) Patent Application Publication
(10) Pub. No.: US 2023/0092478 A1
(43) Pub. Date: Mar. 23, 2023

(54) Device and Method for Weld Root Hardening Determination Compensated for Variations in Distance Between Sensor and Sample

(71) Applicant: ExxonMobil Upstream Research Company, Spring, TX (US)

(72) Inventors: Gaoxiang WU, Houston, TX (US); Jamey A. FENSKE, Spring, TX (US); Chih-Hsiang KUO, Spring, TX (US); Michael BELOTA, Spring, TX (US)

(21) Appl. No.: 17/931,221

(22) Filed: Sep. 12, 2022

Related U.S. Application Data
(60) Provisional application No. 63/261,365, filed on Sep. 20, 2021.

(51) Int. Cl. G01N 27/80; G01N 33/207

(52) U.S. Cl. CPC G01N 27/80; G01N 33/207

(57) ABSTRACT

A device and method for weld root hardening determination compensated for variations in distance between sensor and sample are disclosed. A sensor is used to determine hardness of a weld for weld fabrication quality control. Because of irregular weld protrusion geometry, there may be variations in the tip of the sensor and the surface, resulting in inconsistent measurements. To compensate, one or both of a positional compensation or a software compensation are performed. Positional compensation mechanically moves the tip of the sensor to within a predetermined range of the surface. Software compensation may at least partly compensate for the variation by using one part of the generated sensor data (such as the 1^{st} harmonic signal) in order to modify another part of the generated sensor data (such as the 3^{rd} harmonic signal). In this way, the sensor determination of hardness of the weld may be less dependent on the variations.

### CROSS-REFERENCE TO RELATED APPLICATIONS

This application claims the benefit of U.S. Provisional Application No. 63/261,365, filed Sep. 20, 2021, the disclosure of which is herein incorporated by reference in its entirety.

### FIELD OF THE INVENTION

The present application relates to material inspection, and more particularly to nondestructive material inspection.

### BACKGROUND OF THE INVENTION

This section is intended to introduce various aspects of the art, which may be associated with exemplary embodiments of the present disclosure. This discussion is believed to assist in providing a framework to facilitate a better understanding of particular aspects of the present disclosure. Accordingly, it should be understood that this section should be read in this light, and not necessarily as admissions of prior art.

Pipeline inspection gauges (PIGs) have been used as a tool to perform nondestructive pipeline inspection to detect anomalies and defects in a pipe, such as cracks and hard spots. For example, spots that have a hardness greater than a certain hardness threshold (e.g., greater than 250 Vickers Hardness number) may crack when exposed to certain environments. Responsive to determining that the hardness is greater than the hardness threshold, the hard spot may be repaired (e.g., remove the weld and repair it). The most commonly used technologies include magnetic flux leakage (MFL), ultrasonic crack detection tool (UT), and electromagnetic acoustic transducer (EMAT) that couple electromagnetic energy with a mechanic wave. Similarly, welds are non-destructively inspected using technologies including magnetic particle testing, ultrasonic testing, and eddy current testing. Still another type of non-destructive testing relies on the nonlinear nature of the magnetic response in ferromagnetic materials, such as disclosed in U.S. Patent Application Publication No. 2019/0145931A1, U.S. Patent Application Publication No. 2019/0145932A1, U.S. Patent Application Publication No. 2019/0145933A1, U.S. Patent Application Publication No. 2019/0145934A1, each of which are incorporated by reference herein in their entirety.

These inspection technologies may be based on the principle that the anomalies and defects possess some material properties that are detectably different from that of the bulk material. For example, devices that rely on the nonlinear nature of the magnetic response may be used for local hard zone detection on ferromagnetic metal surfaces. This hardness sensing capability may be qualitative with no precision defined for the measurements. Further, confirmation of weld hardness may be key for quality control of weld fabrication, as discussed above. In this way, the PIGs may evaluate hard spots and/or other suitable material conditions and inhomogeneities (e.g., in pipeline steel or other suitable materials) for nondestructive inspection of pipeline, piping, steel plates, welded structures and welds of different types that can include, but are not limited to, girth welds, fillet welds, lap welds and butt welds, which may be valuable in determining material integrity (e.g., pipeline integrity) as well as material and weld quality.

### SUMMARY OF THE INVENTION

In one or some embodiments, a device for determining one or more material conditions of a sample is disclosed. The device includes: a sensor configured to interrogate the sample with an input time varying magnetic field and to generate sensor data indicative of magnetic responses or acoustic responses over time from the sample; and one or both of a positional compensation or software compensation in order to at least partly compensate for variations in distance between the sensor and a surface of the sample.

In one or some embodiments, a device configured to determine one or more material conditions of a sample. The device includes: a sensor configured to interrogate the sample with an input time varying magnetic field and to generate sensor data indicative of magnetic responses or acoustic responses over time from the sample; a positional compensation mechanism configured to positionally compensate for variations between the sensor and a surface of the sample such that the variations are within a predetermined amount; and a software compensation algorithm configured to further compensate for the variations within the predetermined amount.

In one or some embodiments, a method for determining one or more material conditions of a sample is disclosed. The method includes: interrogating the sample with an input time varying magnetic field from a sensor in order to generate sensor data indicative of magnetic responses or acoustic responses over time from the sample; and performing a software compensation on at least a part of the sensor data in determining one or more qualities of the sample, the software compensation at least partly compensating for variations in distance between the sensor and a surface of the sample.

### BRIEF DESCRIPTION OF DRAWINGS

The present application is further described in the detailed description which follows, in reference to the noted plurality of drawings by way of non-limiting examples of exemplary implementations, in which like reference numerals represent similar parts throughout the several views of the drawings. In this regard, the appended drawings illustrate only exemplary implementations and are therefore not to be considered limiting of scope, for the disclosure may admit to other equally effective embodiments and applications.

FIG. 1 illustrates a schematic of a probe fixed above a protruded weld surface.

FIG. 2A is a schematic diagram of one example device shown having a transmitting coil and a pickup coil on the same side of a material.

FIG. 2B is a schematic diagram of another example device.

FIG. 3 is a flow chart of an example methodology.

FIG. 4A is a graph illustrating the 3^{rd} harmonic signal without signal correction.

FIG. 4B is a graph illustrating the 3^{rd} harmonic signal with signal correction.

FIG. 5 is a graph illustrating signal differentiation between a soft weld and a hard weld across a temperature range between 50° C. to 300° C.

FIG. 6 is a graph illustrating results of lift-off calibration for different microstructures.

FIG. 7 is a diagram of an exemplary computer system that may be utilized to implement the methods described herein.

### DETAILED DESCRIPTION OF THE INVENTION

The methods, devices, systems, and other features discussed below may be embodied in a number of different forms. Not all of the depicted components may be required, however, and some implementations may include additional, different, or fewer components from those expressly described in this disclosure. Variations in the arrangement and type of the components may be made without departing from the spirit or scope of the claims as set forth herein. Further, variations in the processes described, including the addition, deletion, or rearranging and order of logical operations, may be made without departing from the spirit or scope of the claims as set forth herein.

It is to be understood that the present disclosure is not limited to particular devices or methods, which may, of course, vary. It is also to be understood that the terminology used herein is for the purpose of describing particular embodiments only, and is not intended to be limiting. As used herein, the singular forms “a,” “an,” and “the” include singular and plural referents unless the content clearly dictates otherwise. Furthermore, the words “can” and “may” are used throughout this application in a permissive sense (i.e., having the potential to, being able to), not in a mandatory sense (i.e., must). The term “include,” and derivations thereof, mean “including, but not limited to.” The term “coupled” means directly or indirectly connected. The word “exemplary” is used herein to mean “serving as an example, instance, or illustration.” Any aspect described herein as “exemplary” is not necessarily to be construed as preferred or advantageous over other aspects. The term “uniform” means substantially equal for each sub-element, within about ±10% variation.

As used herein, “hydrocarbon management” or “managing hydrocarbons” includes any one, any combination, or all of the following: hydrocarbon extraction; hydrocarbon production, (e.g., drilling a well and prospecting for, and/or producing, hydrocarbons using the well; and/or, causing a well to be drilled, e.g., to prospect for hydrocarbons); hydrocarbon exploration; identifying potential hydrocarbon-bearing formations; characterizing hydrocarbon-bearing formations; identifying well locations; determining well injection rates; determining well extraction rates; identifying reservoir connectivity; acquiring, disposing of, and/or abandoning hydrocarbon resources; reviewing prior hydrocarbon management decisions; and any other hydrocarbon-related acts or activities, such activities typically taking place with respect to a subsurface formation. The aforementioned broadly include not only the acts themselves (e.g., extraction, production, drilling a well, etc.), but also or instead the direction and/or causation of such acts (e.g., causing hydrocarbons to be extracted, causing hydrocarbons to be produced, causing a well to be drilled, causing the prospecting of hydrocarbons, etc.). Hydrocarbon management may include reservoir surveillance and/or geophysical optimization. For example, reservoir surveillance data may include, well production rates (how much water, oil, or gas is extracted over time), well injection rates (how much water or CO₂ is injected over time), well pressure history, and time-lapse geophysical data. As another example, geophysical optimization may include a variety of methods geared to find an optimum model (and/or a series of models which orbit the optimum model) that is consistent with observed/measured geophysical data and geologic experience, process, and/or observation.

As used herein, “obtaining” data generally refers to any method or combination of methods of acquiring, collecting, or accessing data, including, for example, directly measuring or sensing a physical property, receiving transmitted data, selecting data from a group of physical sensors, identifying data in a data record, and retrieving data from one or more data libraries.

As used herein, terms such as “continual” and “continuous” generally refer to processes which occur repeatedly over time independent of an external trigger to instigate subsequent repetitions. In some instances, continual processes may repeat in real time, having minimal periods of inactivity between repetitions. In some instances, periods of inactivity may be inherent in the continual process.

If there is any conflict in the usages of a word or term in this specification and one or more patent or other documents that may be incorporated herein by reference, the definitions that are consistent with this specification should be adopted for the purposes of understanding this disclosure.

As discussed in the background, various types of non-destructive testing, such as non-destructive testing relying on the nonlinear nature of the magnetic response in ferromagnetic materials, may be applied to weld hardness monitoring. As discussed above, the non-destructive testing may be used in a variety of applications, such as in: (1) measuring hard spots in plates (e.g., steel plate); (2) weld root hardness (e.g., for girth welds); (3) identifying pipeline strain (e.g., measuring the physical stretch in the pipeline responsive to an event, such as an earthquake, to determine which sections of the pipeline have been stressed/strained); and/or (4) long seam hardness (e.g., in vintage pipes). The predetermined hardness threshold may vary based on the specific application. Further, the non-destructive testing may identify one or more types of anomalies, flaws, or qualities in samples such as any one, any combination, or all of: the hardness of welds and changes therein; the hardness of the material and changes therein used to produce or in pipes or similar structures; the grade of the material used to produce or in pipes or similar structures; the type of weld; the presence of a material phase in the material (e.g., the presence of a hard steel phase such as martensite or bainite in carbon steel, nonhysteretic material phases in hysteretic ferromagnetic materials and hysteretic magnetic material phases in nonhysteretic materials); the presence of hard spots in the material; the presence of metal loss or cracks in the material (e.g., stress corrosion cracks); the presence of defects in the material, and combinations thereof.

For example, in the pipeline and oil and gas industry, carbon and low alloy steels may be used in the construction of pipelines. Generally, soft ferrite is the dominating phase in these steels. A hard phase such as the martensite phase could form when these steels have been subjected to rapid quenching from high temperature (e.g., above 900° C.) to room temperature. Such rapid quenching could happen either intentionally or accidentally during common steel processing or welding, such as steel component (e.g., steel metal plates, bolts, forgings, castings, and the like) manufacturing or electrical resistance seam welding process, for example. The presence of hard phase in steels can be particularly disadvantageous because it is more susceptible to cracking and failures than the soft ferrite phase. As a result, a nondestructive technique to detect and differentiate martensite from ferrite in the steels is valuable to the industry. As discussed below, the device may be used to detect the difference in magnetic hysteresis parameters between ferrite and martensite phases and differentiate them.

Thus, there may be one or more challenges in the non-destructive testing such as any one, any combination, or all of: signal interference caused by irregular weld protrusion geometry (e.g., surface tension resulting from cooling during welding process may create a protrusion on the weld face); signal-to-hardness calibration; measurement accuracy; and high-temperature interference to magnetic signal.

In one or some embodiments, the non-destructive testing device is configured to perform a compensation to at least partly compensate for variations in distance between the sensor and the surface of the sample. The compensation may comprise a software compensation and/or a positional compensation. With regard to the software compensation, the testing device may include a sensor configured to interrogate the sample with an input time varying magnetic field and to generate sensor data indicative of magnetic responses or acoustic responses over time from the sample. The software compensation may modify the sensor data to at least partly compensate for variations in the distance between at least a part of the sensor (such as the tip of the sensor) and the surface of the sample. For example, the sensor data, induced by eddy currents, may comprise different parts (such as different harmonics) with one part of the sensor data (e.g., the 1^{st} harmonic signal) being used to modify another part of the sensor data (e.g., the 3^{rd} harmonic signal). In particular, the 1^{st} harmonic signal may be used to modify the 3^{rd} harmonic signal thereby generating a corrected 3^{rd} harmonic signal. In a specific embodiment, the only harmonic used to compensate the 3^{rd} harmonic signal is the 1^{st} harmonic signal.

The correction may be performed using one or more equations or one or more look-up tables of values, with inputs of the 1^{st} harmonic signal and the 3^{rd} harmonic signal and output of the corrected 3^{rd} harmonic signal. In one or some embodiments, the equations or look-up tables may be dependent on the material of the sample. For example, a first material type may be correlated to a first equation (with inputs of the Pt harmonic signal and the 3^{rd} harmonic signal and output of the corrected 3^{rd} harmonic signal) and a second material type may be correlated to a second equation. In practice, the type of material (e.g., whether first material type or second material type) may be determined, such as by user input or by the device automatically sensing. Responsive to the type of material determined, the correlated equation or look-up table may be accessed in order to obtain the corrected 3^{rd} harmonic signal (e.g., responsive to user input indicating the sample is of the first material type, the first equation correlated to the first material type may be accessed in order to generate the corrected 3^{rd} harmonic signal).

Alternatively, or in addition, a positional compensation may be performed. Prior to performing any software compensation, the device may be calibrated using the positional compensation. The positional compensation may be used in order to set the height in order to obtain an optimal signal. In one example, the height set may be based on an average distance from the sample. Thereafter, to manage specific protrusions in the sample, software compensation may be performed. In this regard, the positional compensation may be used for a general calibration and the software compensation may be performed in order to at least partly compensate for height differences in the well bead.

In one or some embodiments, an indicator of variations in the distance between the sensor and the surface of the sample is generated, and at least a part of the device (such as the tip of the sensor) is positionally moved responsive to the indication. In a specific embodiment, the indicator comprises at least a part of the sensor data that may be used in order to determine whether the tip of the sensor is in an acceptable position relative to the surface. In one or some embodiments, the 1^{st} harmonic signal comprises the indicator. In practice for the calibration, the 1^{st} harmonic signal is compared with a predetermined 1^{st} harmonic signal that is considered acceptable (indicating that the tip of the sensor is in an acceptable position relative to the surface). In one or some embodiments, the predetermined 1^{st} harmonic signal (against which the 1^{st} harmonic signal (from the sensor) is compared against) is selected based on the material of the surface. In particular, a first material may have an associated first predetermined Pt harmonic signal and a second material may have an associated second predetermined 1^{st} harmonic signal. In practice, the system may include a data structure, such as a look-up table, which correlates predetermined 1^{st} harmonic signals with different materials. Thus, in practice, the system may access the desired predetermined 1^{st} harmonic signal based on the material of the surface. Responsive to determining that the 1^{st} harmonic signal is outside of an acceptable range from the predetermined 1^{st} harmonic signal, the tip of the sensor is moved (e.g., either manually or via a motor). After moving the tip of the sensor, the process is repeated: the 1^{st} harmonic signal is compared again to the predetermined 1^{st} harmonic signal; if the 1^{st} harmonic signal is outside of the acceptable range from the predetermined 1^{st} harmonic signal, the tip of the sensor is moved. The process may be repeatedly performed for a predetermined number of cycles or until the 1^{st} harmonic signal is within the acceptable range from the predetermined 1^{st} harmonic signal.

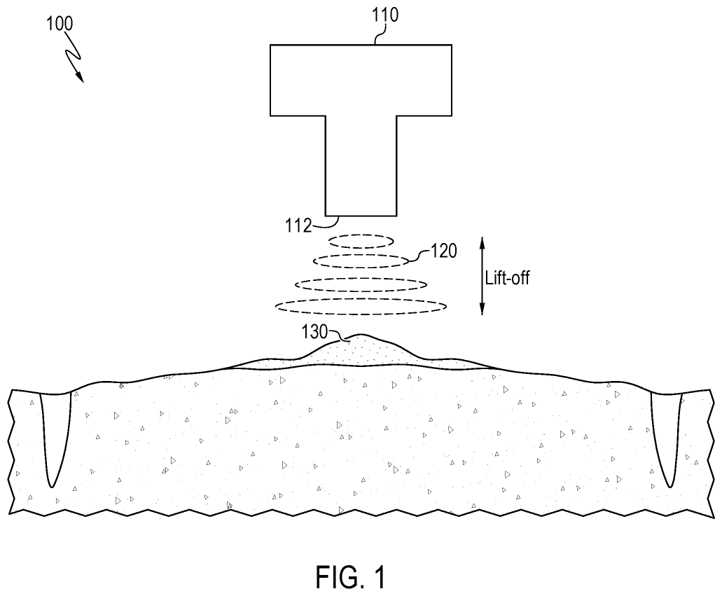

Referring to the figures, FIG. 1 illustrates a schematic 100 of a probe 110 fixed above a protruded weld surface 130 in which a tip 112 of the probe 110 emits electromagnetic radiation 120. As discussed further below, the 3^{rd} harmonic signal (interchangeably termed the 3^{rd} harmonic magnetic signal) induced by eddy current reflects hardness of a ferromagnetic metal surface. Use of the 3^{rd} harmonic signal to determine hardness may be limited, particularly for non-flat surfaces, due to interference by lift-off distance (e.g., the distance between the sensor tip 112 and the metal surface) as lift-off distance may vary across the weld protrusion surface with the probe 110 fixed at a specific height level, as shown in FIG. 1. As discussed in more detail below, the effect on the 3^{rd} harmonic signal due to variation in lift-off distance may be at least partly compensated by a compensation factor, such as a correction based on the 1St harmonic signal.

For background, a linear response function of a magnetic material may be given by the following equation:

B(x)=μ₀(H(x)+M(x))=F(H(x)) (1)

where H(x) is the applied field magnetic field strength (units of ampere/meter) which can vary with position (x) in space, M(x) is the magnetization (units of ampere/meter) which depends on position (x) as well as the initial magnetization state of the material, to is the magnetic permeability constant (unit of henry/meter), B(x) is the magnetic flux density (units of Tesla) which can vary with position (x) in space, and F(H(x)) is a function that depends linearly on H(x). Hereafter, B(x), H(x), M(x) and F(H(x)) are referred to as B, H, M and F(H) respectively, and/or B(t), H(t), M(t) and F(H(t)) if the corresponding parameters are varying with time. This linear dependence is the type of response seen in static magnetic fields. Current inspection tools such as magnetic flux leakage (MFL), and electromagnetic acoustic transducer (EMAT) tools are configured to respond to a function that depends primarily linearly on H. It should be noted that for a ferromagnetic material this dependence may be complicated. When the applied field is time varying, the linear operator no longer describes the relationship between the applied magnetic field and the magnetization. The magnetic flux density B(t) in a ferromagnetic material with an applied time varying magnetic field H(t) can be approximated by a linear operator along with a time integral of a series of nonlinear functions:

B(t)=F(H(t))+∫_{-∞}⁰F₁(H(t+τ))dτ+∫_{-∞}⁰F₂(H²(t+τ))dτ+∫_{-∞}⁰F₃(H³(t+τ))dτ+. . . =F(H(t))+∫_{-∞}⁰Σₙ₌₁^{∞}Fₙ(Hⁿ(t+τ))dτ (2).

The function F₂ gives rise to a second order nonlinear response, the function F₃ gives rise to a third order nonlinear response, and the function Fₙ gives rise to an nth order nonlinear response. The time integral ∫_{-∞}⁰Fₙ(Hⁿ(t+τ))dτ represents that the magnetic flux density B(t) depends on the history of function Fₙ(Hⁿ(t)).

These nonlinear responses may be utilized to provide a better way to characterize material conditions and characterize inhomogeneities in materials. Examples of material conditions and inhomogeneities in materials include, but are not limited to, the hardness of welds, the hardness of the material used to produce or in pipes or similar structures, the grade of the material used to produce or in pipes or similar structures, the type of weld, the hardness of the material, the presence of a material phase in the material (e.g., the presence of a hard steel phase such as martensite or bainite in carbon steel, nonhysteretic material phases in hysteretic ferromagnetic materials, and hysteretic magnetic material phases in nonhysteretic materials), the presence of hard spots in the material, the presence of metal loss or cracks in the material (e.g., stress corrosion cracks), the presence of defects in the material, and combinations thereof. If the applied field H(t) is sinusoidal and varies sinusoidally with a frequency ω, then the second order response may vary as 2ω, the third order response may vary as 3ω and the nth order response may vary as nω. If the applied field has an arbitrary time dependence, then the nonlinear response may be extracted from an analysis of the time dependence of signals that can arise from magnetization and the magnetic flux density (B(t) in Eq. 1). In some cases, this may be done by Fourier analysis of the time dependence of signals arising from magnetization and the magnetic flux density (B(t) in Eq. 1). In some instances, the nonlinear response may be directly characterized from the time dependence of signals arising from magnetization and the magnetic flux density (B(t) in Eq. 1).

The following provides an understanding of the nonlinear magnetic response in hysteretic ferromagnetic materials. Certain embodiments may provide a fast, simple, and general way to detect material conditions and inhomogeneities of a sample being studied. Certain embodiments may not require a built-in ferromagnetic core, and may thus be calibrated in air environment to provide precise background signal. The methodology also allows calibration in environments other than air (for example, samples immersed in oil).

The nonlinear response of the magnetic flux density (B(t) in Eq. 1) in an applied time varying magnetic field gives rise to a number of responses that may be detected. These responses track the time dependence of the magnetic flux density created from the applied time varying magnetic field and the nonlinear responses arise from the hysteretic responses of the magnetization and the magnetic flux density (B(t) in Eq. 1). Both theoretically and experimentally, it is shown that a symmetric hysteretic response leads to odd numbers of harmonics, while an asymmetric hysteretic response leads to even numbers of harmonics. A symmetric hysteresis response usually connects to, but is not limited to, ferromagnetic materials, and an asymmetric hysteresis response usually connects to, but is not limited to, the residual magnetization state in the hysteretic materials, embodiments can also be applied to detect magnetization state of hysteretic materials. One embodiment includes interrogation of a sample with a time varying magnetic field from a magnetic transmitter and detection of the magnetic flux density (B(t) in Eq. 1) with a magnetic sensor that is in proximity to the sample. A variation of this embodiment includes the incorporation of a DC magnetic field that biases the magnetization. Another variation includes the measurement of a sample with a residual magnetization. Yet another variation includes measurement of a sample that has been degaussed. A different embodiment includes interrogation of a sample with a time varying magnetic field from a magnetic transmitter and detection of the magnetic flux density (B(t) in Eq. 1) and a nonlinear magneto-acoustic response (e.g., similar to EMAT), but looks at the nonlinear spectra of acoustic signal. A variation of this embodiment includes the incorporation of a DC magnetic field that biases the magnetization. Another variation includes the measurement of a sample with a residual magnetization. Yet another variation measurement of a sample that has been degaussed.

The general principle of the nonlinear magnetic response relies on applying a time varying magnetic field H(t) to a sample and detecting a response. This principle will be illustrated from the case in which the time varying magnetic field is an AC magnetic modulation H_{AC}(\vec{r}, t)=H₁(\vec{r})e^{iωt} with a spatially varying magnetization field H₁(\vec{r}) and angular frequency ω=2πf. Such AC modulation can be achieved by a time varying electrical current J_{f}=Jₒ (\vec{r})e^{iωt} (considering Ampere's law):

$$∇ \times {H}_{1} = {J}_{f} + \frac{\partial D}{\partial t} , \qquad ( 3 )$$

and the second term

$$\frac{\partial D}{\partial L} \qquad ( 4 )$$

is negligible in a frequency range of interest.

$$\vert \frac{\partial D}{\partial t} \vert ∼ \vert - i ωϵ E \vert ≪ σ \vert E \vert ) . \qquad ( 5 )$$

A DC magnetizing field H_{DC}(\vec{r}) may also be applied by a DC electrical current or permanent magnet, and leads to a total field generated by the source.: Hₛ(\vec{r},t)=H_{DC}(\vec{r})+H_{AC}(\vec{r})e^{iωt}. For ferromagnetic materials such as carbon steel and other ferritic phase in steels, the relative permeability μᵣ that connects field B and H is a hysteretic and nonlinear operator. Therefore, the primary magnetic field Bₛ(\vec{r}, t)=μₒμᵣHₛ(\vec{r}, t) would be nonlinear inside the ferromagnetic materials and may be described as a Taylor series:

Bₛ(\vec{r}, t)=Σ_{n=-∞}^{n=∞}Bₙ(\vec{r})e^{inωt} (6).

With Faraday's law:

$$∇ \times {E}_{2} ( \vec{r} , t ) = - \frac{\partial {B}_{s} ( \vec{r} , t )}{\partial t} , \qquad ( 7 )$$

the induced electric field in steel E₂(\vec{r}, t) and the resulting Eddy current J_{eddy}(\vec{r}, t)=σE₂(\vec{r}, t) are both nonlinear, as the conductivity in a steel is normally a scalar and linear operator. The Eddy current is only distributed around the surface of conductive materials with a skin depth of:

$${d}_{p} = \sqrt{\frac{1}{π f μ σ}} , \qquad ( 8 )$$

and it generates a secondary magnetizing field H₂(\vec{r}, t) from Ampere's law ∇×H₂=J_{eddy}=σE₂. As a result, the secondary magnetizing field:

$$\vert {H}_{2} \vert ∞ - σ \frac{\partial {B}_{s}}{\partial t} \qquad ( 9 )$$

would contain similar nonlinear information as the primary field Bₛ(\vec{r}, t).

Different ferromagnetic materials have different hysteresis curves and magnetic responses, and would result in different nonlinear harmonic coefficient Bₙ(\vec{r}) under the same magnetic modulation. The difference in the harmonic coefficients may be measured with two methods:
1. Nonlinear magnetic detection: The total magnetizing field is nonlinear, and may be measured by a magnetic sensor at a point A in air: Hₜₒₜₐₗ(\vec{r}_{A}, t)=H_{DC}(\vec{r}_{A})+H_{AC}(\vec{r}_{A})e^{iωt}+H₂(\vec{r}_{A}, t).
2. Nonlinear magnetoacoustic detection. With a large constant DC magnetic field B_{DC}(\vec{r}), a strong Lorentz body force f(\vec{r}, t)=J_{eddy}(\vec{r}, t)×B_{DC}(\vec{r}) takes place and launches a time-varying mechanic wave. Such magnetoacoustic response is also nonlinear.

Finally considered is the generation of different nonlinear harmonics under sinusoidal modulation J_{f}=Jₒ(\vec{r})e^{iωt}, and in this case all the nonlinear effects originate from Bₛ(\vec{r}, t)=μₒμᵣHₛ(\vec{r}, t). When the local hysteresis B-H loop is symmetric inside the hysteretic materials, Bₛ(\vec{r}, t) reverse its direction after half a period

$${B}_{s} ( \vec{r} , t ) = - {B}_{s} ( \vec{r} , t + \frac{T}{2} ) .$$

This normally happens at near zero magnetization. With Taylor expansion from equation (1), the symmetry constrain suggests:

$${\sum }_{n = - ∞}^{n = ∞} {B}_{n} ( \vec{r} ) {e}^{in ω t} = {\sum }_{n = - ∞}^{n = ∞} {B}_{n} ( \vec{r} ) {e}^{in ω ( t + \frac{T}{2} )} \qquad ( 10 )$$

and Bₙ(\vec{r})=(−1)⁽ⁿ⁺¹⁾Bₙ(\vec{r}). Therefore, for even numbers of n, the harmonic coefficient Bₙ(\vec{r})=0. In other words, a symmetric B-H curve may prohibit the generation of even number harmonics and only allows odd number of harmonics. In contrast, if the B-H loop is asymmetric,

$${B}_{s} ( \vec{r} , t ) \neq - {B}_{s} ( \vec{r} , t + \frac{T}{2} )$$

and all Taylor coefficient Bₙ(\vec{r}) in the expansion could exist. In other words, an asymmetric B-H curve allows for both odd and even numbers of harmonics.

Generating the sensor data may comprise interrogating the hysteretic ferromagnetic material by applying one at a time varying magnetic field. Optionally, an additional DC magnetic field can be applied. Optionally, a degaussing magnetic field can be applied. Optionally, the sample may have a residual magnetization. A DC magnetic field is a magnetic field that is not varying over time, and a degaussing magnetic field is a time-varying magnetic field that is used to eliminate residual magnetization of a material. The time varying magnetic response or acoustic response may be detected. The methodology may also include determining a time dependent non-linear characteristic of the received magnetic field or acoustic response and correlating the time dependent nonlinear characteristic of the received magnetic response or acoustic response to one or more material conditions of the material.

Interrogating the hysteretic ferromagnetic material with an input time varying magnetic field may include, but is not limited to, utilizing at least one magnetic transmitter that generates a time varying magnetic field and placing the magnetic transmitter at a nearby location to the interrogated sample. For example, an example proximity (or nearby location) for the magnetic transmitter is 1 cm to the surface of the interrogated sample; a more preferred nearby location for the magnetic transmitter is 0.2 cm or less to the surface of the interrogated sample; an even more preferred nearby location for the magnetic transmitter is in direct contact on the surface of the interrogated sample.

The time varying magnetic field may include, but is not limited to, a combination of sinusoidal wave, square wave, triangular wave and symmetric and asymmetric pulses. In certain embodiments, a preferred time varying magnetic field can include sinusoidal wave with peak ampli-tude ranging from 0.01 milliTesla to 1 Tesla, and frequency ranging from 1 Hz to 1 MHz. A more preferred time varying magnetic field can include sinusoidal wave with peak ampli-tude s from 0.1 milliTesla to 10 milliTesla, and frequency ranging from 100 Hz to 100 kHz. For examining 4140 carbon steel materials, and other carbon steel materials made with the Thermo-Mechanical Controlled Processing (TMCP) such as X60 and/or X65 carbon steel, a preferred time varying magnetic field can include sinusoidal wave with peak amplitude ranging from 0.01 milliTesla to 1 Tesla, and frequency ranging from 1 Hz to 1 MHz; a more preferred time varying magnetic field can include sinusoidal wave with peak amplitudes from 0.1 milliTesla to 10 milliTesla, and frequency ranging from 100 Hz to 100 kHz; an even more preferred time varying magnetic field can include sinusoidal wave with peak amplitudes from 0.1 milliTesla to 10 milliTesla, and frequency ranging from 8 kHz to 100 kHz; an even more preferred time varying magnetic field can include sinusoidal wave with peak amplitudes from 0.5 milliTesla to 5 milliTesla, and frequency ranging from 8 kHz to 100 kHz.

Similar to the common practice in other non-destructive inspection tools, one familiar with the technique may optimize the time varying magnetic field by calibrating the nonlinear magnetic response and/or the size of 3^{rd} harmonics with respect to frequency range, amplitude range, and material phases.

The magnetic transmitter may include, but is not limited to, a device to generate the time varying magnetic field, such as a transmitting coil, a translating/rotating magnet such as Neodymium magnet, ceramic magnet, electro-magnet, or a superconducting magnet. In certain embodiments, a preferred magnetic transmitter can include a transmitting coil with an outer diameter between 2 mm to 10 cm, number of turns between 1 to 100,000 and an inductance between 0.001 mH to 1000 mH. In certain embodiments, a more preferred magnetic transmitter can include a transmit-ting coil with an outer diameter between 5 mm to 5 cm, number of turns between 10 to 1000 and an inductance between 0.01 mH to 100 mH. In certain embodiments, a more preferred magnetic transmitter can include a transmitting coil with an outer diameter of 1 inch (25.4 mm), 100 turns, and an inductance of L˜0.25 mH. In certain embodiments, a smaller-diameter magnetic transmitter can be used to generate inspection results with higher lateral spatial resolution. In certain embodiments, an even more preferred magnetic transmitter can include one or more coils with their diameters smaller than 1-inch to improve the lateral spatial resolution of the inspection results.

Detecting a magnetic response or an acoustic response may include, but is not limited to, utilizing at least one magnetic sensor or acoustic sensor configured to receive a magnetic response or acoustic response, respectively, and to convert the magnetic response or acoustic response into magnetic response signals or acoustic response signals. In one or some embodiments, the magnetic sensor is located in a region near the magnetic transmitter. In one embodiment, the distance between the magnetic sensor and the magnetic transmitter is less than 50 meters, preferably less than 10 meters, preferably less than 1 meter, preferably less than 10 centimeters, preferably less than 1 centimeters, preferably less than 1 millimeter, and even more preferably in direct contact with each other.

A magnetic response may include, but is not limited to, a spatially varying magnetic field produced by the interrogated material as a result of input time varying magnetic field and any additional magnetic fields. A magnetic sensor can include, but is not limited to, a device to receive the magnetic response from at least one point or averaged over a sensing area, and convert the magnetic response to a digital or analogue signal that can be interpreted by a computer or observer, such as pickup coils, Hall sensors, Fluxgate magnetometers, Cesium atomic magnetometers or superconducting SQUID magnetometers. In certain embodiments, a preferred magnetic sensor can include a sensing coil with an outer diameter between 2 mm to 10 cm, number of turns between 1 to 100,000 and an inductance between 0.001 mH to 1000 mH. In certain embodiments, a more preferred magnetic transmitter can include a transmitting coil with an outer diameter between 5 mm to 5 cm, number of turns between 10 to 1000 and an inductance between 0.01 mH to 100 mH. In certain embodiments, an even more preferred magnetic sensor can include a sensing coil with an outer diameter of 1 inch, 100 turns, and an inductance of L˜0.25 mH. In certain embodiments, a smaller-diameter magnetic sensor can be used to generate inspection results with higher lateral spatial resolution. In certain embodiments, a more preferred magnetic sensor can include one or more coils with their diameters smaller than 1-inch to improve the lateral spatial resolution of the inspection results. In one embodiment, the magnetic sensor is chosen so that it may respond sufficiently fast to record at least the signal arising from the second order nonlinear effect, in a more preferred embodiment, the magnetic sensor is chosen so that it can respond sufficiently fast to record at least the signal arising from the third order nonlinear effect, and in an even more preferred embodiment, the magnetic sensor is chosen so that it can respond sufficiently fast to record at least the signal arising from the fifth order non-linear effect.

An acoustic response may include, but is not limited to, a mechanical motion produced by the interrogated material as a result of input time varying magnetic field and any additional magnetic fields. An acoustic sensor can include, but is not limited to, a device to receive the acoustic response from at least one point or averaged over a sensing area, and convert the acoustic response to a digital or analog signal that can be interpreted by a computer or observer, such as piezoelectric acoustic transducer, microphone, seismometer, or geophone. In certain embodiments, a preferred acoustic sensor can include a ceramic piezoelectric acoustic transducer with a diameter of 1.2 cm and a resonance frequency of 500 kHz. In one embodiment, the acoustic sensor is chosen so that it can respond sufficiently fast to record at least the signal arising from the second order nonlinear effect, in a more preferred embodiment, the acoustic sensor is chosen so that it can respond sufficiently fast to record at least the signal arising from the third order nonlinear effect and in an even more preferred embodiment, the sensor is chosen so that it can respond sufficiently fast to record at least the signal arising from the fifth order nonlinear effect.

Determining the time dependent non-linear characteristic may include performing a frequency domain analysis such as power spectral density analysis of the received magnetic response or acoustic response to create power spectral density data. In certain embodiments, determining the time dependent non-linear characteristic may include determining one or more harmonic peak values of the power spectral density data.

Determining the one or more harmonic peak values may include determining one or more harmonic coefficients of the spectral density data. For example, determining the one or more harmonic coefficients and/or peak values may include determining odd harmonic coefficients and/or peak values of the spectral density data.

In certain embodiments, determining the odd harmonic coefficients and/or peak values may include determining 3^{rd} and/or 5^{th} harmonics of the spectral density data. Correlating the time dependent nonlinear characteristics can include comparing and correlating the 3^{rd} and/or 5^{th} harmonics to the one or more material conditions of an interrogated sample. In certain embodiments, a large 3^{rd} harmonics of the spectral density data, ranging from 10⁻⁶ or above after normalization, correlate to a material condition that include, but is not limited to, the presence of ferrite or pearlite carbon steel phases in an interrogated sample; a small 3^{rd} harmonics of the spectral density data, ranging from 10⁻⁸ to 10⁻⁶ after normalization, correlate to a material condition that include, but is not limited to, the presence of hard steel phase such as martensite or lath bainite carbon steel phases, or nonhysteretic material such as air gap in an interrogated sample.

The interrogated sample may include, but is not limited to, a test material composed of at least one material phase with one or more material conditions. Examples of material conditions and inhomogeneities in materials include, but are not limited to, the hardness of welds, the hardness of the material used to produce or in pipes or similar structures, the grade of the material used to produce or in pipes or similar structures, the type of weld, the hardness of the material, the presence of a material phase in the material, the presence of hard spots in the material, the presence of metal loss or cracks in the material, the presence of defects in the material, and combinations thereof. For example, the one or more material conditions may include at least one material phase of the hysteretic ferromagnetic material or the nonhysteretic material. In certain embodiments, the hysteretic ferromagnetic material may include, but is not limited to steel, nickel, cobalt, and some of their alloys, such as a variety of carbon steels. The nonhysteretic material may include, but is not limited to air, aluminum, austenitic stainless steel, duplex stainless steel, and high manganese steel. The material phase may include, but is not limited to, at least one of austenite, martensite, ferrite, pearlite, bainite, lath bainite, acicular ferrite, and quasipolygonal ferrite with different chemical compositions and/or crystallographic orientations. The inhomogeneities of a sample may include, but are not limited to, a test material composed of more than one material phase. Nonlimiting examples of inhomogeneities are hard spots and/or cracks/defects, e.g., in a steel pipe.

In one or some embodiments, a non-transitory computer readable medium may include instructions for performing any suitable method as described herein and/or any suitable portion(s) thereof. For example, the method can include generating a time varying magnetic field and detecting a magnetic response or acoustic response signal over time from a pickup coil, determining a time dependent non-linear characteristic of the received magnetic field or acoustic response, and correlating the time dependent nonlinear characteristic of the received magnetic response or acoustic response to one or more material conditions of the material. Any other suitable portions of any embodiment of a method as described herein can be included additionally or alternatively.

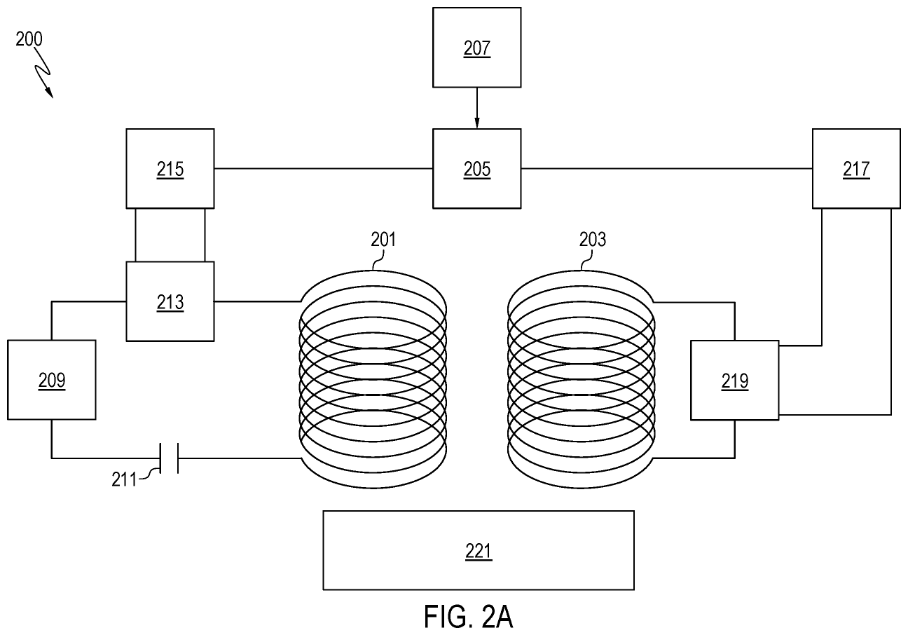

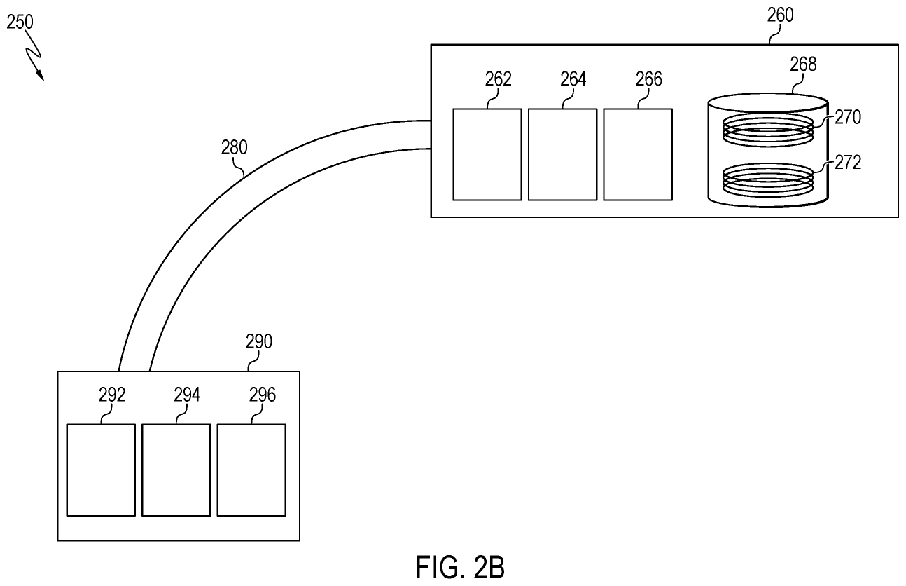

Referring to FIG. 2A, a device 200 for detecting one or more material conditions of a hysteretic ferromagnetic material (e.g., a sample 221 comprising a hysteretic ferromagnetic material) may include a transmitting coil 201 configured to output an interrogation magnetic field, a pickup coil 203 or acoustic transducer (e.g., as described in more detail below) configured to receive a magnetic response or acoustic response, respectively, and to convert the magnetic response or acoustic response into magnetic signals or acoustic response signals. The device 200 may include a processor 205 configured to execute any suitable method, e.g., as described hereinabove and/or any suitable portion(s) thereof. For example, the processor 205 may execute the software compensation described herein.

In one or some embodiments, the device 200 may include an output device 207 (e.g., a display) configured to indicate to a user the one or more conditions of the material. The device 200 may include any other suitable signal processing components (e.g., one or more digitizers, a current meter, a signal generator, one or more bandpass filters, one or more pre-amplifiers or amplifiers, etc.) as appreciated by those having ordinary skill in the art. The output device 207 may include, but is not limited to, an indicator, which implies to notify one or more nearby users for appropriate immediate, real-time actions, and the users can directly observe the indicator. The output device 207 may also include, but is not limited to, a device for communicating to users, which also implies notify users for appropriate immediate, real-time actions, but the users may be at a remote location, and the communication may through wired or wireless routes. The output device for later retrieval and post-processing and analysis.

Carbon steels may be key materials in the pipeline and oil & gas industry. Generally, all the carbon steels compose of multiple material phases. Ferrite (soft phase of carbon steel) may be a key material phase in the carbon steels. Hard phase, such as martensite or lath bainite may form in the steels when they have been rapidly quenched from high temperature (for example, from 900° C.) to room temperature, which may happen during steel mill plate manufacture or an electrical resistance seam welding process. The presence of hard steel such as martensite or lath bainite phases may be particularly precarious as it is more susceptible to failures and cracking compared to soft ferrite phase. As a result, a carbon steel sample composed of ferrite and martensite are tested herein, since the application to pipelines is a good example of where such devices may be used. Any other suitable materials and applications are contemplated herein.

In the embodiment shown, a voltage or current signal may be generated through the signal generator 209 (e.g., a sinusoidal wave of frequency f). With current passing through, the transmitting coil 201 is used as a magnetic transmitter to generate a modulating magnetic field. The transmitting coil 201 used to produce data below includes an outer diameter of about 1 inch, 100 turns, and an inductance of L˜0.25 mH. The electrical impedance of a transmitting coil is Z_{coil}=RᵢₙₜₑᵣₙₐₗiωL. Typically, the internal resistance Rᵢₙₜₑᵣₙₐₗ of a coil is relatively small (<112 for the coil), while the imaginary inductive term increases proportionally with frequency. Any other suitable coil with any suitable characteristics may be used.

To minimize the impedance effect of the inductor and maximize the output current, a capacitor C 211 may be used to change the total impedance to:

$${Z}_{c o i l} = {R}_{internal} + i ω L + \frac{1}{i ω C} , \qquad ( 11 )$$

while the imaginary term may be cancelled out when:

$$i ω L + \frac{1}{i ω C} = 0 or C = i ω L + \frac{1}{{ω}^{2} L} . \qquad ( 12 )$$

Frequencies from 1 kHz to 100 kHz may be used in generating the data below, and different capacitances may be used at different frequencies to ensure that:

$$C ∼ \frac{1}{{ω}^{2} L} . \qquad ( 13 )$$

For the same transmitting coil 201. The current passing through the transmitting coil 201 may be measured with a current meter 213 and recorded with a first digitizer 215.

To detect the magnetic response from nearby materials, a magnetic sensor such as the pickup coil 203 may be used to measure time varying magnetic signal. The voltage generated through the pickup coil 203 is:

$$E = - N \frac{dB}{dt} . \qquad ( 14 )$$

A, which is related to number of turns N of the pickup coil 203, time derivative of local magnetic field and cross-section area of the loop A. This voltage may be measured through a second digitizer 217, for example. An optional pre-amplifier and/or bandpass filter 219 may be utilized between pickup coil 203 and the second digitizer 217, e.g., to enhance weak signal or detect specific frequency components in the measured signal if necessary. After receiving the waveforms of the transmitting current and pickup voltage from both digitizers, PSD analysis may be performed by the processor 205 in real time to extract nonlinear coefficients and/or peak values of the testing materials.

The transmitting coil 201, pickup coil 203, and the interrogated material may be arranged in any suitable configuration. In FIG. 2A, the transmitting coil 201 and pickup coil 203 are placed to the same side of the interrogated material (e.g., ferromagnetic plate), and this configuration may be readily applied to conventional PIG for nondestructive pipeline inspection. Alternatively, the transmitting coil 201 and pickup coil 203 placed on opposite sides of interrogated material.

Accordingly, as shown in FIG. 2A, the device 200 may be configured for use on a single side of the interrogated material. In certain embodiments, the one or more conditions of the material to be determined may include a material phase, for example.

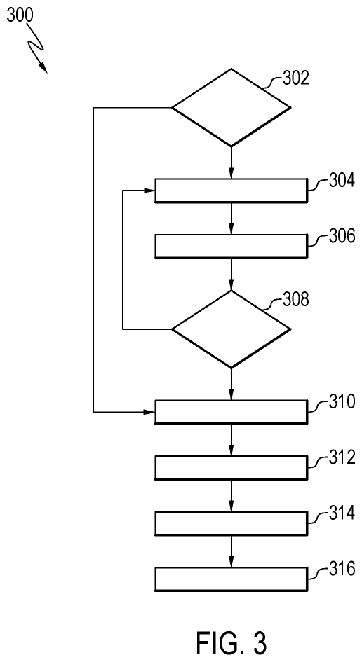

FIG. 2B is a schematic diagram 250 of another example device including sensor portion resident in well 260, cable 280, and backend electronics 290. Sensor portion resident in well 260 may be configured to be placed or moved within a well in order to examine welds therein, and may include sensor control electronics 262, motor 264, structure to move position of at least a part of the sensor 266 (such as the sensor tip), and coil housing 268 (including sensing coil 270 (also known as a pickup coil) and transducing coil 272 (also known as a transmitting coil), positioned on top of one another). In practice, sensor control electronics 262 may receive commands from backend electronics 260 and control one or more of motor 264, structure to move position of at least a part of the sensor 266, sensing coil 270 and transducing coil 272. As discussed below with regard to FIG. 3, positional compensation may be performed by motor 264 and structure to move position of at least a part of the sensor 266. In one or some embodiments, the structure to move position of at least a part of the sensor 266 may comprise a screw mechanism that may mechanically adjust the tip of the probe further away and/or closer to the surface.

Further, sensor portion resident in well 260 may send sensor data, generated responsive to activation of transducing coil 272 and sensed by sensing coil 270, to backend electronics 290 for processing by processor 292 in combination with memory 294. As discussed below, part of the processing may include the software compensation to at least partly compensate for variations in distance between the sensor and the surface of the sample. The results of the processing may be output on display 296.

FIG. 3 is a flow chart 300 of an example methodology. At 302, it is determined whether to perform mechanical adjustment. If not, flow chart 300 moves to 310. If so, flow chart 300 moves to 304 in order to obtain the 1^{st} harmonic signal. Responsive to obtaining the 1^{st} harmonic signal, movement of at least a part of the device (such as the tip of the sensor) is controlled to adjust the position of the sensor (such as to adjust the tip of the sensor). In one or some embodiments, the processor 292 on the backend electronics 290 may receive the 1^{st} harmonic signal, compare the 1^{st} harmonic signal to a threshold, and responsive to the comparison indicating that sensor should be moved positionally, send a command to sensor control electronics 262 to control motor 264 to move at least a part of the sensor. After movement, at 308, it is determined whether additional mechanical adjustment is necessary. If so, flow chart 300 moves back to 304. If not, flow chart 300 moves to 310.

At 310, the material of the sample is determined. As discussed above, the material of the sample may be determined responsive to user input (e.g., the user input as to the material is stored in memory 294 for later access by processor 292) or may be determined automatically via analysis of the sample. At 312, a lookup is performed to determine software compensation for the material of the sample. As discussed above, the software compensation may be dependent on the material. In this regard, a lookup table may be used correlating different materials to different software corrections. In practice, the processor may access the lookup table, with the determined material in order to access the correlated software correction.

At 314, the sensor obtains sensor data, including the 1^{st} harmonic signal and the 3^{rd} harmonic signal. At 316, the software compensation (using the software compensation accessed at 312) of the 3^{rd} harmonic signal is performed based the 1^{st} harmonic signal.

In one or some embodiments, the 1^{st} harmonic signal may respond strongly to lift-off distance resulted from the irregular weld protrusion geometry, while barely responding to hardness changes on the metal surface. Therefore, a signal correction mechanism may utilize the 1^{st} harmonic response to the lift-off distance, so that hardness of girth welds (as indicated by the corrected 3^{rd} harmonic signal) may be measured with correction to minimize the signal interference caused by irregular weld geometries. The 3^{rd} harmonic signal correction may be achieved in a variety of ways. In one example, the equation below may be used for the correction:

Corrected 3^{rd} Harmonic=3^{rd} Harmonic/(a*log(1^{st} Harmonic)+b) (15)

where a and b are constants calculated based on lift-off calibration using a base metal. In this regard, a and b may be re-calculated when materials and test parameters are changed. Specifically, the base material may be calibrated to obtain correlation between the 3^{rd} harmonic signal and the lift-off distance, so 3^{rd} Harmonic/3^{rd} Harmonic (@1^{st} Harmonic=0.1) may be curve-fitted as a natural log function of 1^{st} Harmonic:

3^{rd} Harmonic/3^{rd} Harmonic (@1^{st} Harmonic=0.1)=a*log(1^{st} Harmonic)+b (16).

It is noted that a and b may be two curve fitting-determined constants. In one or some embodiments, the 3^{rd} Harmonic (@1^{st} Harmonic=0.1) is an experimentally determined value. 0.1 may be selected to be the reference 1^{st} harmonic point because it is the value when lift-off distance is controlled within a predetermined range (e.g., between 1.50 mm to 1.75 mm) and it deemed sufficient based on the data set analyzed. The reference point may be selected based on different base materials and preferences.

Thus, in a first step, positional compensation is performed. In practice, the sensor generates 1^{st} harmonic data, compares the 1^{st} harmonic data to the experimentally determined value (in the example, 0.1), and modifies the position of at least a part of the sensor (e.g., the tip of the sensor) based on the comparison. In one embodiment, the sensor may automatically (and optionally iteratively) generate the 1^{st} harmonic data, compare the 1^{st} harmonic data to the experimentally determined value, and modify the position of at least a part of the sensor (e.g., sensor 266) based on the comparison. The iterations may be performed until the value of the 1^{st} harmonic data is within a predetermined range of the experimentally determined value. Alternatively, the positional compensation may be performed at least partly manually. For example, the sensor may: generate 1^{st} harmonic data; compare the 1^{st} harmonic data to the experimentally determined value; and output the comparison (or an indicator of the comparison, such as an indication of how much to move the tip of the sensor). As one example, the sensor may output a number and a direction of turns of the screw mechanism, which may comprise the structure to move position of at least a part of the sensor 266. In response to the output, the user may move the position of at least a part of the sensor (e.g., turn the screw mechanism). Further, in manual mode, the sensor may perform a single iteration or multiple iterations.

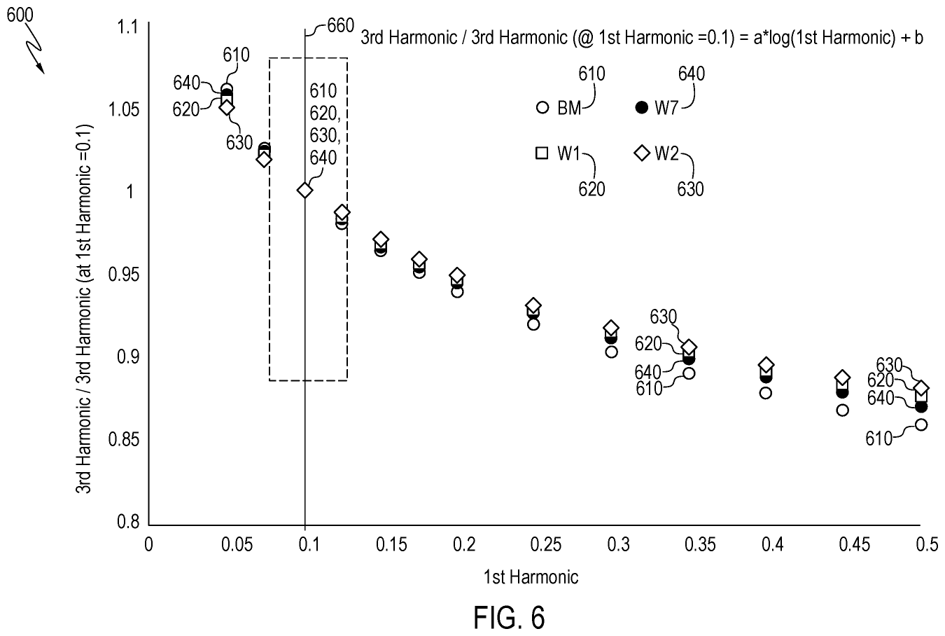

After the positional compensation is performed, the tip of the sensor is adjusted such that the 1^{st} harmonic is at least within a certain range (as identified by 650 in FIG. 6) for the ratio 3^{rd} Harmonic/3^{rd} Harmonic (@1^{st} Harmonic=0.1). In this way, the software compensation may perform more minor compensations within 650 due to deviations of the 1^{st} harmonic from the experimentally determined value (in the example, 0.1). In particular, the sensed 1^{st} harmonic and Eq. (16) may be used to determine the ratio 3^{rd} Harmonic/3^{rd} Harmonic (@1^{st} Harmonic=0.1). In turn, Eq. (15) may be used to determine the corrected 3^{rd} harmonic.

FIG. 6 illustrates a graph 600 of the lift-off calibration for different microstructures, including for the base metal (BM) 610 and different welds, W1 (620), W2 (630), and W7 (640). In practice, the welds may be composed of slightly different material than the BM. As such, the curves, as illustrated in graph 600 for the BM 610 versus the different welds, W1 (620), W2 (630), and W7 (640), are different. As shown in the graph 600, there is convergence at approximately 0.1 for the 1^{st} Harmonic (shown at line 660). As such, the value selected may be @1^{st} Harmonic of 0.1 (at the common crossing point), or may be selected in a range around 0.1. It is noted that for different base materials, the common crossing point may be different than 0.1.

Graph 600 further includes an area of interest 650, illustrated by rectangle. As discussed above, position adjustment may be performed prior to software adjustment. For example, the positional adjustment may comprise movement so that the sensor is within the area of interest 650. In particular, responsive to sensing the 1^{st} harmonic and comparing to a predetermined harmonic (which may be based on look-up table, as discussed above), the position of the tip of the sensor may be moved, such that the sensor, from a positional standpoint, is within the area of interest 650. In this way, the positional adjustment may comprise a coarser calibration in order to be within or proximate to the area of interest 650, with the software adjustment comprising a finer calibration in order to further reduce effects of liftoff issues, such as illustrated in FIG. 1.

In effect, the software adjustment may compensate for liftoff issues. As shown above, the 1^{st} harmonic at a certain value (e.g., at 0.1 as shown in FIG. 6) is a common point at which the 3^{rd} harmonic values for the different curves converge (see curves for BM 610, W1 (620), W2 (630), and W7 (640)). As such, the correction may be based on the divergence from the 1^{st} harmonic common point, which is then translated into a correction for the 3^{rd} harmonic. In effect, the correction seeks to modify the value of the 3^{rd} Harmonic to what it would be when the 1st harmonic is at the common point (e.g., 1^{st} harmonic=0.1).

By way of the example illustrated in FIG. 6, when the 1^{st} harmonic=0.1, the ratio (3^{rd} harmonic/3^{rd} harmonic (at 1^{st} harmonic=0.1))=1, resulting in no correction (the corrected 3^{rd} harmonic equals the 3^{rd} harmonic as measured (which is approximately 5.0)). In contrast, where the 1^{st} harmonic is measured at 0.095 (slightly to the left of line 660), the ratio is approximately 1.02 (1/0.095), resulting in the measured 3^{rd} harmonic (of approximately 5.1) being corrected to the value of the 3^{rd} harmonic (when the 1^{st} harmonic=0.1) of 5.0. In this way, the corrected 3^{rd} harmonic may be the same (or nearly the same) for different values of the 1^{st} harmonic.

In this way, the positional compensation (acting as a coarse compensation) may move the tip of the surface of the sensor such that the variations between the tip of the sensor and the surface are within a predetermined amount (e.g., within the area of interest 650). Within the area of interest (in which the multiple curves are more closely aligned), a finer compensation, via the software compensation, may be performed.

In one or some embodiments, analyzing/performing positional compensation may be performed one, some, or every time the sensor is moved to a new weld. Thus, responsive to moving to the new weld, the device may determine/move to the optimal average height (e.g., see 302, 304, 306, 308), and thereafter perform the scanning/software compensation (e.g., see 312, 314, 316).

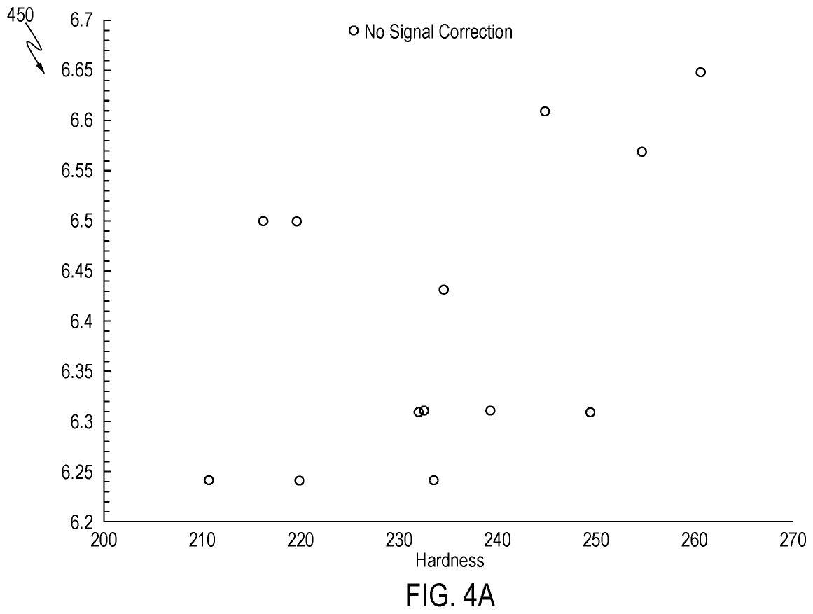

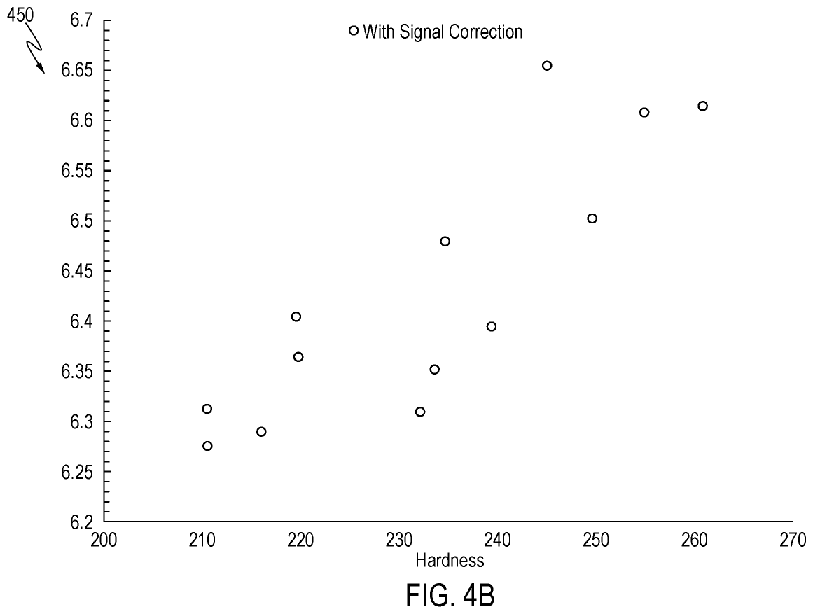

Measurement data, with the disclosed correction, demonstrates a detection rate of ˜90% with a hardness accuracy of +/−12.5. FIG. 4A is a graph 400 illustrating the 3^{rd} harmonic signal as a function of weld root hardness without signal correction, while FIG. 4B is a graph 450 illustrating the 3^{rd} harmonic signal as a function of weld root hardness with signal correction. Based on the comparison of FIG. 4A with FIG. 4B, a much stronger correlation may be observed after the signal correlation applied. To improve spatial resolution of the signals, a small-size Eddy-current probe (˜10 mm in diameter, size comparable to width of girth welds) was utilized.

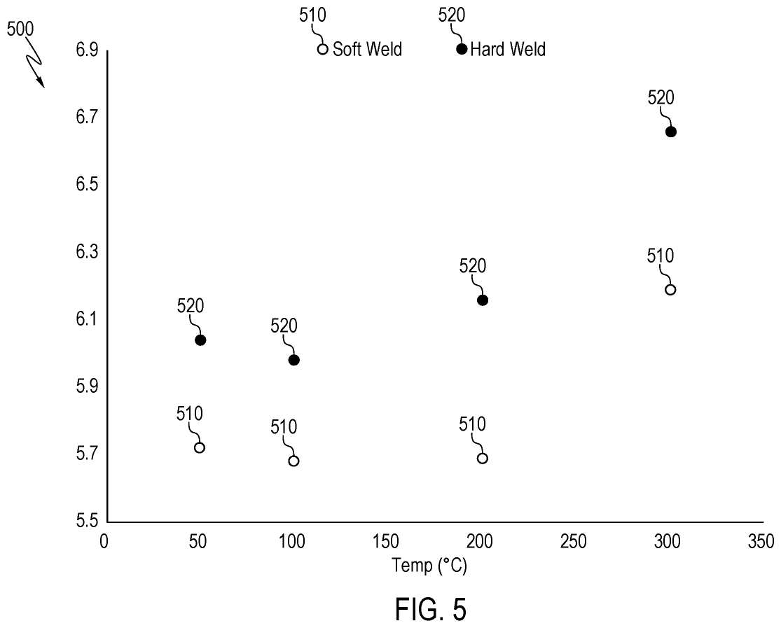

In addition, it was determined that the signal differentiation signified by the value differences due to hardness difference did not diminish with increasing temperature, at least up to 300° C., as shown in the graph 500 in FIG. 5 illustrating signal differentiation between a soft weld 510 and a hard weld 520 across a temperature range between 50° C. to 300° C. Thus, the disclosed methodology for girth weld root hardness monitoring may be still applicable at high temperature.

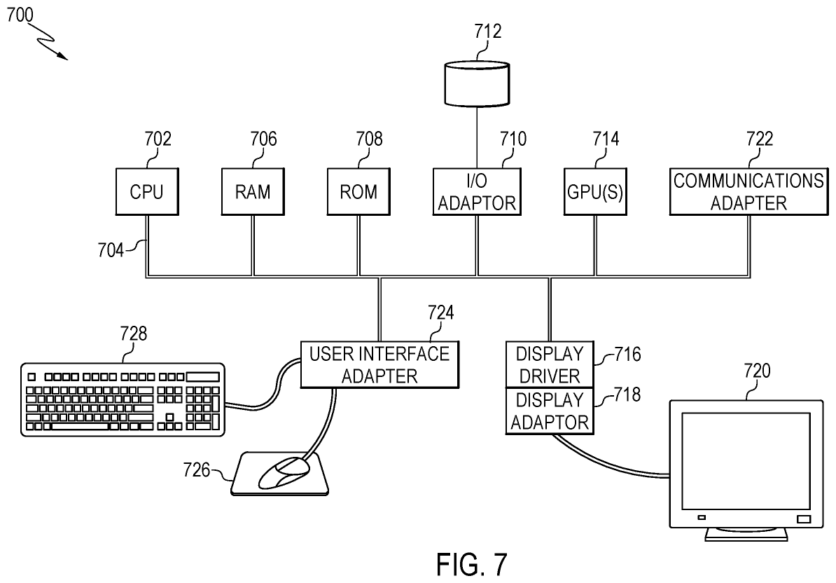

In all practical applications, the present technological advancement must be used in conjunction with a computer, programmed in accordance with the disclosures herein. For example, FIG. 7 is a diagram of an exemplary computer system 700 that may be utilized to implement methods described herein. A central processing unit (CPU) 702 is coupled to system bus 704. The CPU 702 may be any general-purpose CPU, although other types of architectures of CPU 702 (or other components of exemplary computer system 700) may be used as long as CPU 702 (and other components of computer system 700) supports the operations as described herein. Those of ordinary skill in the art will appreciate that, while only a single CPU 702 is shown in FIG. 7, additional CPUs may be present. Moreover, the computer system 700 may comprise a networked, multi-processor computer system that may include a hybrid parallel CPU/GPU system. The CPU 702 may execute the various logical instructions according to various teachings disclosed herein. For example, the CPU 702 may execute machine-level instructions for performing processing according to the operational flow described.

The computer system 700 may also include computer components such as non-transitory, computer-readable media. Examples of computer-readable media include computer-readable non-transitory storage media, such as a random-access memory (RAM) 706, which may be SRAM, DRAM, SDRAM, or the like. The computer system 700 may also include additional non-transitory, computer-readable storage media such as a read-only memory (ROM) 708, which may be PROM, EPROM, EEPROM, or the like. RAM 706 and ROM 708 hold user and system data and programs, as is known in the art. The computer system 700 may also include an input/output (I/O) adapter 710, a graphics processing unit (GPU) 714, a communications adapter 722, a user interface adapter 724, a display driver 716, and a display adapter 718.

The I/O adapter 710 may connect additional non-transitory, computer-readable media such as storage device(s) 712, including, for example, a hard drive, a compact disc (CD) drive, a floppy disk drive, a tape drive, and the like to computer system 700. The storage device(s) may be used when RAM 706 is insufficient for the memory requirements associated with storing data for operations of the present techniques. The data storage of the computer system 700 may be used for storing information and/or other data used or generated as disclosed herein. For example, storage device(s) 712 may be used to store configuration information or additional plug-ins in accordance with the present techniques. Further, user interface adapter 724 couples user input devices, such as a keyboard 728, a pointing device 726 and/or output devices to the computer system 700. The display adapter 718 is driven by the CPU 702 to control the display on a display device 720 to, for example, present information to the user such as subsurface images generated according to methods described herein.

The architecture of computer system 700 may be varied as desired. For example, any suitable processor-based device may be used, including without limitation personal computers, laptop computers, computer workstations, and multi-processor servers. Moreover, the present technological advancement may be implemented on application specific integrated circuits (ASICs) or very large scale integrated (VLSI) circuits. In fact, persons of ordinary skill in the art may use any number of suitable hardware structures capable of executing logical operations according to the present technological advancement. The term “processing circuit” encompasses a hardware processor (such as those found in the hardware devices noted above), ASICs, and VLSI circuits. Input data to the computer system 700 may include various plug-ins and library files. Input data may additionally include configuration information.

Preferably, the computer is a high-performance computer (HPC), known to those skilled in the art. Such high-performance computers typically involve clusters of nodes, each node having multiple CPU's and computer memory that allow parallel computation. The models may be visualized and edited using any interactive visualization programs and associated hardware, such as monitors and projectors. The architecture of system may vary and may be composed of any number of suitable hardware structures capable of executing logical operations and displaying the output according to the present technological advancement. Those of ordinary skill in the art are aware of suitable supercomputers available from Cray or IBM or other cloud computing based vendors such as Microsoft, Amazon.

The above-described techniques, and/or systems implementing such techniques, can further include hydrocarbon management based at least in part upon the above techniques, including using the device in one or more aspects of hydrocarbon management. For instance, methods according to various embodiments may include managing hydrocarbons based at least in part upon the device and data representations constructed according to the above-described methods. In particular, such methods may use the device to evaluate various welds in the context of drilling a well.

It is intended that the foregoing detailed description be understood as an illustration of selected forms that the invention can take and not as a definition of the invention. It is only the following claims, including all equivalents which are intended to define the scope of the claimed invention. Further, it should be noted that any aspect of any of the preferred embodiments described herein may be used alone or in combination with one another. Finally, persons skilled in the art will readily recognize that in preferred implementation, some, or all of the steps in the disclosed method are performed using a computer so that the methodology is computer implemented. In such cases, the resulting physical properties model may be downloaded or saved to computer storage.

The following example embodiments of the invention are also disclosed.

Embodiment 1: A device for determining one or more material conditions of a sample, the device comprising:
a sensor configured to interrogate the sample with an input time varying magnetic field and to generate sensor data indicative of magnetic responses or acoustic responses over time from the sample; and
one or both of a positional compensation or software compensation in order to at least partly compensate for variations in distance between the sensor and a surface of the sample.

Embodiment 2: The device of embodiment 1,
further comprising a processor in communication with the sensor and configured to perform the software compensation in determining one or more qualities of the sample, the software compensation at least partly compensating for variations in distance between the sensor and a surface of the sample.

Embodiment 3: The device of any of embodiments 1 or 2,
wherein the processor is configured to determine the one or more qualities at least partly compensated for the variations in distance between the sensor and the surface of the sample by:
using at least a part of the sensor data in order to modify another part of the sensor data thereby generating modified sensor data that at least partly compensates for the variations in the distance between a tip of the sensor and the surface of the sample; and
using the modified sensor data in order to determine the one or more qualities.

Embodiment 4: The device of any of embodiments 1-3,
wherein the one or more qualities comprise hardness.

Embodiment 5: The device of any of embodiments 1-4,
wherein the sensor data comprises a 1^{st} harmonic signal and a 3^{rd} harmonic signal induced by eddy currents;
wherein the processor is configured to generate the modified sensor data by modifying the 3^{rd} harmonic signal based on the 1^{st} harmonic signal in order to generate a corrected 3^{rd} harmonic signal; and
wherein the processor is configured to use the modified sensor data in order to determine the hardness by analyzing the corrected 3^{rd} harmonic signal in order to determine the hardness of the sample.

Embodiment 6: The device of any of embodiments 1-5,
wherein the processor is configured to modify the 3^{rd} harmonic signal based on both the 1^{st} harmonic signal and a type of material for the sample in order to generate the corrected 3^{rd} harmonic signal.

Embodiment 7: The device of any of embodiments 1-6,
wherein the processor includes a look up table correlating types of materials to corrections to modify the 3^{rd} harmonic signal to generate the corrected 3^{rd} harmonic signal.

Embodiment 8: The device of any of embodiments 1-7, further comprising:
a processor in communication with the sensor and configured to generate an indicator for compensation of the variations in distance between the sensor and the surface of the sample; and
structure to positionally move the sensor, based on the indicator, in order to at least partly compensate for the variations in distance between the sensor and the surface of the sample.

Embodiment 9: The device of any of embodiments 1-8,
wherein the sensor data comprises a 1^{st} harmonic signal induced by eddy currents; and
wherein the indicator for compensation is based on the 1^{st} harmonic signal.

Embodiment 10: The device of any of embodiments 1-9,
further comprising a motor configured to mechanically move a tip of the sensor relative to the surface of the sample; and
wherein the processor, based on the Pt harmonic signal, is configured to control the motor in order to move tip of the sensor relative to the surface of the sample in order to at least partly compensate for the variations in distance between the sensor and the surface of the sample.

Embodiment 11: The device of any of embodiments 1-10,
wherein the processor is further configured to perform the software compensation in determining one or more qualities of the sample, the software compensation of at least partly compensating for variations in distance between the sensor and a surface of the sample.

Embodiment 12: A device configured to determine one or more material conditions of a sample, the device comprising:
a sensor configured to interrogate the sample with an input time varying magnetic field and to generate sensor data indicative of magnetic responses or acoustic responses over time from the sample;
a positional compensation mechanism configured to positionally compensate for variations between the sensor and a surface of the sample such that the variations are within a predetermined amount; and
a software compensation algorithm configured to further compensate for the variations within the predetermined amount.

Embodiment 13: The device of embodiment 12,
further comprising a processor in communication with the sensor and configured to execute the software compensation algorithm in determining one or more qualities of the sample;
wherein the positional compensation mechanism is based on at least a part of the sensor data; and
wherein the software compensation algorithm uses the at least a part of the sensor data in order to modify another part of the sensor data thereby generating modified sensor data that at least partly compensates for the variations in the distance between a tip of the sensor and the surface of the sample.

Embodiment 14: A method for determining one or more material conditions of a sample, the method comprising:
interrogating the sample with an input time varying magnetic field from a sensor in order to generate sensor data indicative of magnetic responses or acoustic responses over time from the sample; and
performing a software compensation on at least a part of the sensor data in determining one or more qualities of the sample, the software compensation at least partly compensating for variations in distance between the sensor and a surface of the sample.

Embodiment 15: The method of embodiment 14,
wherein determining the one or more qualities at least partly compensated for the variations in distance between the sensor and the surface of the sample comprises:
using at least a part of the sensor data in order to modify another part of the sensor data thereby generating modified sensor data that at least partly compensates for the variations in the distance between a tip of the sensor and the surface of the sample; and
using the modified sensor data in order to determine the one or more qualities.

Embodiment 16: The method of any of embodiments 14 or 15,
wherein the one or more qualities comprise hardness.

Embodiment 17: The method of any of embodiments 14-16,
wherein the sensor data comprises a 1^{st} harmonic signal and a 3^{rd} harmonic signal induced by eddy currents;
wherein generating the modified sensor data is performed by modifying the 3^{rd} harmonic signal based on the 1^{st} harmonic signal in order to generate a corrected 3^{rd} harmonic signal; and
wherein using the modified sensor data in order to determine the hardness comprises analyzing the corrected 3^{rd} harmonic signal in order to determine the hardness of the sample.

Embodiment 18: The method of any of embodiments 14-17,
wherein modifying the 3^{rd} harmonic signal is based on both the 1^{st} harmonic signal and a type of material for the sample in order to generate the corrected 3^{rd} harmonic signal.

Embodiment 19: The method of any of embodiments 14-18,
further comprising performing a positional compensation in order to positionally move at least a part of the sensor in order to at least partly compensate for the variations in distance between the sensor and the surface of the sample.

Embodiment 20: The method of any of embodiments 14-19,
wherein the positional compensation is performed prior to the software compensation.

Embodiment 21: The method of any of embodiments 14-20,
wherein the sensor data comprises a 1^{st} harmonic signal and a 3^{rd} harmonic signal induced by eddy currents;
wherein the software compensation comprises:
   accessing a correction factor based on a composition of the sample; and modifying the 3^{rd} harmonic signal based on the 1^{st} harmonic signal and the
   correction factor in order to generate the corrected 3^{rd} harmonic signal; and
wherein the corrected 3^{rd} harmonic signal is used in order to determine hardness of the sample.

What is claimed is:

1. A device for determining one or more material conditions of a sample, the device comprising:
a sensor configured to interrogate the sample with an input time varying magnetic field and to generate sensor data indicative of magnetic responses or acoustic responses over time from the sample; and
one or both of a positional compensation or software compensation in order to at least partly compensate for variations in distance between the sensor and a surface of the sample.

2. The device of claim 1, further comprising a processor in communication with the sensor and configured to perform the software compensation in determining one or more qualities of the sample, the software compensation at least partly compensating for variations in distance between the sensor and a surface of the sample.

3. The device of claim 2, wherein the processor is configured to determine the one or more qualities at least partly compensated for the variations in distance between the sensor and the surface of the sample by:
using at least a part of the sensor data in order to modify another part of the sensor data thereby generating modified sensor data that at least partly compensates for the variations in the distance between a tip of the sensor and the surface of the sample; and
using the modified sensor data in order to determine the one or more qualities.

4. The device of claim 3, wherein the one or more qualities comprise hardness.

5. The device of claim 4, wherein the sensor data comprises a 1^{st} harmonic signal and a 3^{rd} harmonic signal induced by eddy currents;
wherein the processor is configured to generate the modified sensor data by modifying the 3^{rd} harmonic signal based on the 1^{st} harmonic signal in order to generate a corrected 3^{rd} harmonic signal; and
wherein the processor is configured to use the modified sensor data in order to determine the hardness by analyzing the corrected 3^{rd} harmonic signal in order to determine the hardness of the sample.

6. The device of claim 5, wherein the processor is configured to modify the 3^{rd} harmonic signal based on both the 1^{st} harmonic signal and a type of material for the sample in order to generate the corrected 3^{rd} harmonic signal.

7. The device of claim 6, wherein the processor includes a look up table correlating types of materials to corrections to modify the 3^{rd} harmonic signal to generate the corrected 3^{rd} harmonic signal.

8. The device of claim 1, further comprising:
a processor in communication with the sensor and configured to generate an indicator for compensation of the variations in distance between the sensor and the surface of the sample; and
structure to positionally move the sensor, based on the indicator, in order to at least partly compensate for the variations in distance between the sensor and the surface of the sample.

9. The device of claim 8, wherein the sensor data comprises a 1^{st} harmonic signal induced by eddy currents; and
wherein the indicator for compensation is based on the 1^{st} harmonic signal.

10. The device of claim 9, further comprising a motor configured to mechanically move a tip of the sensor relative to the surface of the sample; and
wherein the processor, based on the 1^{st} harmonic signal, is configured to control the motor in order to move tip of the sensor relative to the surface of the sample in order to at least partly compensate for the variations in distance between the sensor and the surface of the sample.

11. The device of claim 9, wherein the processor is further configured to perform the software compensation in determining one or more qualities of the sample, the software compensation of at least partly compensating for variations in distance between the sensor and a surface of the sample.

12. A device configured to determine one or more material conditions of a sample, the device comprising:
a sensor configured to interrogate the sample with an input time varying magnetic field and to generate sensor data indicative of magnetic responses or acoustic responses over time from the sample;
a positional compensation mechanism configured to positionally compensate for variations between the sensor and a surface of the sample such that the variations are within a predetermined amount; and
a software compensation algorithm configured to further compensate for the variations within the predetermined amount.

13. The device of claim 12, further comprising a processor in communication with the sensor and configured to execute the software compensation algorithm in determining one or more qualities of the sample;
wherein the positional compensation mechanism is based on at least a part of the sensor data; and
wherein the software compensation algorithm uses the at least a part of the sensor data in order to modify another part of the sensor data thereby generating modified sensor data that at least partly compensates for the variations in the distance between a tip of the sensor and the surface of the sample.

14. A method for determining one or more material conditions of a sample, the method comprising:
interrogating the sample with an input time varying magnetic field from a sensor in order to generate sensor data indicative of magnetic responses or acoustic responses over time from the sample; and
performing a software compensation on at least a part of the sensor data in determining one or more qualities of the sample, the software compensation at least partly compensating for variations in distance between the sensor and a surface of the sample.

15. The method of claim 14, wherein determining the one or more qualities at least partly compensated for the variations in distance between the sensor and the surface of the sample comprises:
using at least a part of the sensor data in order to modify another part of the sensor data thereby generating modified sensor data that at least partly compensates for the variations in the distance between a tip of the sensor and the surface of the sample; and
using the modified sensor data in order to determine the one or more qualities.

16. The method of claim 15, wherein the one or more qualities comprise hardness.

17. The method of claim 16, wherein the sensor data comprises a 1^{st} harmonic signal and a 3^{rd} harmonic signal induced by eddy currents;
wherein generating the modified sensor data is performed by modifying the 3^{rd} harmonic signal based on the 1^{st} harmonic signal in order to generate a corrected 3^{rd} harmonic signal; and
wherein using the modified sensor data in order to determine the hardness comprises analyzing the corrected 3^{rd} harmonic signal in order to determine the hardness of the sample.

18. The method of claim 17, wherein modifying the 3^{rd} harmonic signal is based on both the 1^{st} harmonic signal and a type of material for the sample in order to generate the corrected 3^{rd} harmonic signal.

19. The method of claim 14, further comprising performing a positional compensation in order to positionally move at least a part of the sensor in order to at least partly compensate for the variations in distance between the sensor and the surface of the sample.

20. The method of claim 19, wherein the positional compensation is performed prior to the software compensation.

21. The method of claim 20, wherein the sensor data comprises a 1^{st} harmonic signal and a 3^{rd} harmonic signal induced by eddy currents;
wherein the software compensation comprises:
accessing a correction factor based on a composition of the sample; and
modifying the 3^{rd} harmonic signal based on the 1^{st} harmonic signal and the correction factor in order to generate the corrected 3^{rd} harmonic signal; and
wherein the corrected 3^{rd} harmonic signal is used in order to determine hardness of the sample.
